(19) United States
(12) Patent Application Publication
(10) Pub. No.: US 2024/0130433 A1
(43) Pub. Date: Apr. 25, 2024

(54) AEROSOL PROVISION SYSTEM AND METHOD

(71) Applicant: Nicoventures Trading Limited, London (GB)

(72) Inventor: Patrick Moloney, London (GB)

(21) Appl. No.: 17/769,550

(22) PCT Filed: Sep. 16, 2020
(86) PCT No.: PCT/GB2020/052249

§ 371 Date: Apr. 14, 2022

(30) Foreign Application Priority Data
Oct. 15, 2019 (GB) 1914951.7

(51) Int. Cl. A24F 40/53; A24F 40/60; A24F 40/65

(52) U.S. Cl. CPC A24F 40/53; A24F 40/60; A24F 40/65

(57) ABSTRACT

An aerosol provision system configured to generate aerosol from an aerosol-generating material for user inhalation comprises a computer. The computer is configured to derive user profile data indicating a user's usage of an aerosol provision system as a function of time, obtain data of a target usage profile indicating a target usage of the aerosol provision system as a function of time, estimate a difference between at least a part of the user profile and a corresponding part of the target usage profile for a predetermined period of time, and adjust one or more operational parameters of the aerosol provision system to at least partially map the user's usage as indicated by the user profile data to the target usage of the aerosol provision system for the predetermined period of time.

### PRIORITY CLAIM

The present application is a National Phase entry of PCT Application No. PCT/GB2020/052249, filed Sep. 17, 2020, which claims priority from GB Application No. 1914951.7, filed Oct. 16, 2019, each of which is hereby fully incorporated herein by reference.

### TECHNICAL FIELD

The present disclosure relates to an aerosol provision system and method.

### BACKGROUND

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Electronic aerosol provision systems such as electronic cigarettes (e-cigarettes) generally contain a reservoir of a source liquid containing a formulation, typically including nicotine, from which an aerosol is generated, e.g. through heat vaporization. An aerosol source for an aerosol provision system may thus comprise a heater having a heating element arranged to receive source liquid from the reservoir, for example through wicking/capillary action. Other source materials may be similarly heated to create an aerosol, such as botanical matter, or a gel comprising an active ingredient and/or flavoring. Hence more generally, the e-cigarette may be thought of as comprising or receiving a payload for heat vaporization.

While a user inhales on the device, electrical power is supplied to the heating element to vaporize the aerosol source (a portion of the payload) in the vicinity of the heating element, to generate an aerosol for inhalation by the user. Such devices are usually provided with one or more air inlet holes located away from a mouthpiece end of the system. When a user sucks on a mouthpiece connected to the mouthpiece end of the system, air is drawn in through the inlet holes and past the aerosol source. There is a flow path connecting between the aerosol source and an opening in the mouthpiece so that air drawn past the aerosol source continues along the flow path to the mouthpiece opening, carrying some of the aerosol from the aerosol source with it. The aerosol-carrying air exits the aerosol provision system through the mouthpiece opening for inhalation by the user.

Usually an electric current is supplied to the heater when a user is drawing/puffing on the device. Typically, the electric current is supplied to the heater, e.g. resistance heating element, in response to either the activation of an airflow sensor along the flow path as the user inhales/draw/puffs or in response to the activation of a button by the user. The heat generated by the heating element is used to vaporize a formulation. The released vapor mixes with air drawn through the device by the puffing consumer and forms an aerosol. Alternatively or in addition, the heating element is used to heat but typically not burn a botanical such as tobacco, to release active ingredients thereof as a vapor/aerosol.

The amount of vaporized/aerosolized payload inhaled by the user will depend at least in part on how long and how deeply the user inhales and, over a period of time, how frequently the user inhales as well. In turn, these user behaviors may be influenced by their mood.

Consequently, it would be useful to provide an aerosol delivery mechanism that was more responsive to the user's mood or behavior.

### SUMMARY

In one aspect of the present disclosure an aerosol provision system is configured to generate aerosol from an aerosol generating material for user inhalation. The aerosol provision system includes a computer configured to derive user profile data indicating a user's usage of the aerosol provision system as a function of time, obtain data of a target usage profile indicating a target usage of the aerosol provision system as a function of time, estimate a difference between at least a part of the user profile and a corresponding part of the target usage profile for a predetermined period of time, and adjust one or more operational parameters of the aerosol provision system to at least partially map the user's usage as indicated by the user profile data to the target usage of the aerosol provision system for the predetermined period of time.

In embodiments, the predetermined period of time starts at one of a calendar day, a disconnection of the aerosol provision system from a charger, a first inhalation in a day, or the current time. In embodiments, the predetermined period of time is one of a period corresponding in length to a detected time dependent pattern in the user profile data, a period corresponding in length to a detected time dependent pattern in the target usage profile, a sampling period of the user profile data, an hour, or a day.

In embodiments, the one or more operational parameters are adjusted to change the amount of an active ingredient delivered per unit volume of air inhaled.

In embodiments, the computer is further configured to at least partially map the user's usage as indicated by the user profile data to the target usage of the aerosol provision system, wherein the mapping distributes the total delivered active ingredient indicated by the target usage profile across the user's usage for the predetermined period of time as indicated by the user profile data.

In embodiments, the computer is further configured to at least partially map the user's usage as indicated by the user profile data to the target usage of the aerosol provision system, wherein the mapping distributes delivery of an active ingredient within user inhalations responsive to a schedule of inhalations within the target usage of the aerosol provision system.

In embodiments, the computer is further configured to at least partially map the user's usage as indicated by the user profile data to the target usage of the aerosol provision system, wherein the mapping distributes the delivery of an active ingredient during a respective user inhalation responsive to the difference between an expected inhalation duration indicated by the user profile data and a corresponding target inhalation duration.

In embodiments, the computer is further configured to receive from a user interface an indication from the user to commence mapping.

In embodiments, the operations of the computer are located within one or more of the aerosol provision system, a remote server operable to communicate with the aerosol provision system, a mobile computing device operable to communicate with the aerosol provision system, and a remote server operable to communicate with a mobile computing device operable to communicate with the aerosol provision system.

In one aspect of the present disclosure, a method of aerosol generation includes deriving user profile data indicating a user's usage of an aerosol provision system as a function of time, obtaining data of a target usage profile indicating a target usage of the aerosol provision system as a function of time, estimating a difference between at least a part of the user profile data and a corresponding part of the target usage profile for a predetermined period of time, and adjusting one or more operational parameters of the aerosol provision system to at least partially map the user's usage as indicated by the user profile data to the target usage of the aerosol provision system for the predetermined period of time.

In one aspect of the present disclosure, a non-transitory computer-readable storage medium storing a computer program comprising computer executable instructions adapted to cause a computer system to perform one or more of the methods performed herein.

It is to be understood that both the foregoing general summary of the disclosure and the following detailed description are provided for the purposes of example, and are not intended to be restrictive of the disclosure. Further aspects are provided in accordance with the claims.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings.

FIG. 1 is a schematic diagram depicting an electronic aerosol/vapor provision system (EVPS), according to an embodiment.

FIG. 2 is a schematic diagram depicting further details of the EVPS of FIG. 1.

FIG. 3 is a schematic diagram depicting further details of the EVPS of FIG. 1.

FIG. 4 is a schematic diagram depicting further details of the EVPS of FIG. 1.

FIG. 5 is a schematic diagram depicting a system comprising the EVPS of FIG. 1 and a remote device, according to an embodiment.

FIG. 6 is a flowchart depicting a method of aerosol delivery, according to an embodiment.

FIG. 7 is a graph depicting actual and target EVPS usage profiles, according to an embodiment.

FIG. 8 is a graph depicting adjustments to an EVPS responsive to the actual and target EVPS usage profiles, according to an embodiment.

FIG. 9 is a graph depicting a modified actual EVPS usage profile, according to an embodiment.

### DETAILED DESCRIPTION

An electronic aerosol provision system and method are disclosed. In the following description, a number of specific details are presented in order to provide a thorough understanding of the embodiments of the present disclosure. It will be apparent, however, to a person skilled of ordinary skill in the art that these specific details need not be employed to practice embodiments of the present disclosure. Conversely, specific details known to the person skilled in the art are omitted for the purposes of clarity where appropriate.

As described above, the present disclosure relates to an aerosol provision system (e.g. a non-combustible aerosol provision system) or electronic vapor provision system (EVPS), such as an e-cigarette. Throughout the following description the term “e-cigarette” is sometimes used but this term may be used interchangeably with (electronic) aerosol/vapor provision system. Similarly, the terms ‘vapor’ and ‘aerosol’ are referred to equivalently herein. Generally, the electronic vapor/aerosol provision system may be an electronic cigarette, also known as a vaping device or electronic nicotine delivery system (END), although it is noted that the presence of nicotine in the aerosolizable material is not a requirement. In some embodiments, a non-combustible aerosol provision system is a tobacco heating system, also known as a heat-not-burn system. In some embodiments, the non-combustible aerosol provision system is a hybrid system to generate aerosol using a combination of aerosolizable materials, one or a plurality of which may be heated. Each of the aerosolizable materials may be, for example, in the form of a solid, liquid or gel and may or may not contain nicotine. In some embodiments, the hybrid system comprises a liquid or gel aerosolizable material and a solid aerosolizable material. The solid aerosolizable material may comprise, for example, tobacco or a non-tobacco product. Meanwhile in some embodiments, the non-combustible aerosol provision system generates a vapor/aerosol from one or more such aerosolizable materials.

Typically, the non-combustible aerosol provision system may comprise a non-combustible aerosol provision device and an article for use with the non-combustible aerosol provision system. However, it is envisaged that articles which themselves comprise a means for powering an aerosol-generating component may themselves form the non-combustible aerosol provision system. In one embodiment, the non-combustible aerosol provision device may comprise a power source and a controller. The power source may be an electric power source or an exothermic power source. In one embodiment, the exothermic power source comprises a carbon substrate which may be energized so as to distribute power in the form of heat to an aerosolizable material or heat transfer material in proximity to the exothermic power source. In one embodiment, the power source, such as an exothermic power source, is provided in the article so as to form the non-combustible aerosol provision. In one embodiment, the article for use with the non-combustible aerosol provision device may comprise an aerosolizable material.

In some embodiments, the aerosol-generating component is a heater capable of interacting with the aerosolizable material so as to release one or more volatiles from the aerosolizable material to form an aerosol. In one embodiment, the aerosol-generating component is capable of generating an aerosol from the aerosolizable material without heating. For example, the aerosol-generating component may be capable of generating an aerosol from the aerosolizable material without applying heat thereto, for example via one or more of vibrational, mechanical, pressurization or electrostatic means.

In some embodiments, the aerosolizable material may comprise an active material, an aerosol forming material and optionally one or more functional materials. The active material may comprise nicotine (optionally contained in tobacco or a tobacco derivative) or one or more other non-olfactory physiologically active materials. A non-olfactory physiologically active material is a material, which is included in the aerosolizable material in order to achieve a physiological response other than olfactory perception. The aerosol forming material may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate. The one or more functional materials may comprise one or more of flavors, carriers, pH regulators, stabilizers, and/or antioxidants.

In some embodiments, the article for use with the non-combustible aerosol provision device may comprise aerosolizable material or an area for receiving aerosolizable material. In one embodiment, the article for use with the non-combustible aerosol provision device may comprise a mouthpiece. The area for receiving aerosolizable material may be a storage area for storing aerosolizable material. For example, the storage area may be a reservoir. In one embodiment, the area for receiving aerosolizable material may be separate from, or combined with, an aerosol-generating area.

Referring now to the drawings, wherein like reference numerals designate identical or corresponding parts throughout the several views.

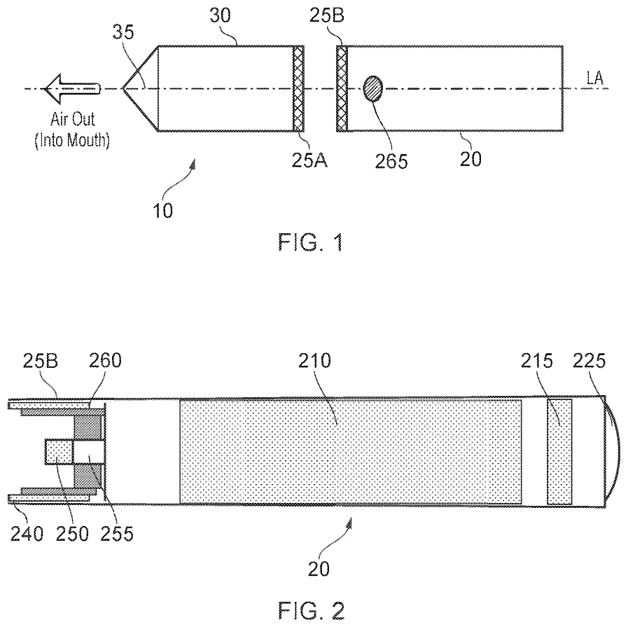

FIG. 1 is a schematic diagram of an electronic vapor/aerosol provision system such as an e-cigarette 10 in accordance with some embodiments of the disclosure (not to scale). The e-cigarette has a generally cylindrical shape, extending along a longitudinal axis indicated by dashed line LA, and comprises two main components, namely a body 20 and a cartomizer 30. The cartomizer includes an internal chamber containing a reservoir of a payload such as for example a liquid comprising nicotine, a vaporizer (such as a heater), and a mouthpiece 35. References to ‘nicotine’ hereafter will be understood to be merely exemplary and can be substituted with any suitable active ingredient. References to ‘liquid’ as a payload hereafter will be understood to be merely exemplary and can be substituted with any suitable payload such as botanical matter (for example tobacco that is to be heated rather than burned), or a gel comprising an active ingredient and/or flavoring. The reservoir may be a foam matrix or any other structure for retaining the liquid until such time that it is required to be delivered to the vaporizer. In the case of a liquid/flowing payload, the vaporizer is for vaporizing the liquid, and the cartomizer 30 may further include a wick or similar facility to transport a small amount of liquid from the reservoir to a vaporizing location on or adjacent the vaporizer. In the following, a heater is used as a specific example of a vaporizer. However, it will be appreciated that other forms of vaporizer (for example, those which utilize ultrasonic waves) could also be used and it will also be appreciated that the type of vaporizer used may also depend on the type of payload to be vaporized.

The body 20 includes a re-chargeable cell or battery to provide power to the e-cigarette 10 and a circuit board for generally controlling the e-cigarette. When the heater receives power from the battery, as controlled by the circuit board, the heater vaporizes the liquid and this vapor is then inhaled by a user through the mouthpiece 35. In some specific embodiments, the body is further provided with a manual activation device 265, e.g. a button, switch, or touch sensor located on the outside of the body.

The body 20 and cartomizer 30 may be detachable from one another by separating in a direction parallel to the longitudinal axis LA, as shown in FIG. 1, but are joined together when the device 10 is in use by a connection, indicated schematically in FIG. 1 as 25A and 25B, to provide mechanical and electrical connectivity between the body 20 and the cartomizer 30. The electrical connector 25B on the body 20 that is used to connect to the cartomizer 30 also serves as a socket for connecting a charging device (not shown) when the body 20 is detached from the cartomizer 30. The other end of the charging device may be plugged into a USB socket to re-charge the cell in the body 20 of the e-cigarette 10. In other implementations, a cable may be provided for direct connection between the electrical connector 25B on the body 20 and a USB socket.

The e-cigarette 10 is provided with one or more holes (not shown in FIG. 1) for air inlets. These holes connect to an air passage through the e-cigarette 10 to the mouthpiece 35. When a user inhales through the mouthpiece 35, air is drawn into this air passage through the one or more air inlet holes, which are suitably located on the outside of the e-cigarette. When the heater is activated to vaporize the nicotine from the cartridge, the airflow passes through, and combines with, the generated vapor, and this combination of airflow and generated vapor then passes out of the mouthpiece 35 to be inhaled by a user. Except in single-use devices, the cartomizer 30 may be detached from the body 20 and disposed of when the supply of liquid is exhausted (and replaced with another cartomizer if so desired).

It will be appreciated that the e-cigarette 10 shown in FIG. 1 is presented by way of example, and various other implementations can be adopted. For example, in some embodiments, the cartomizer 30 is provided as two separable components, namely a cartridge comprising the liquid reservoir and mouthpiece (which can be replaced when the liquid from the reservoir is exhausted), and a vaporizer comprising a heater (which is generally retained). As another example, the charging facility may connect to an additional or alternative power source, such as a car cigarette lighter.

FIG. 2 is a schematic (simplified) diagram of the body 20 of the e-cigarette 10 of FIG. 1 in accordance with some embodiments of the disclosure. FIG. 2 can generally be regarded as a cross-section in a plane through the longitudinal axis LA of the e-cigarette 10. Note that various components and details of the body, e.g. such as wiring and more complex shaping, have been omitted from FIG. 2 for reasons of clarity.

The body 20 includes a battery or cell 210 for powering the e-cigarette 10 in response to a user activation of the device. Additionally, the body 20 includes a control unit (not shown in FIG. 2), for example a chip such as an application specific integrated circuit (ASIC) or microcontroller, for controlling the e-cigarette 10. The microcontroller or ASIC includes a CPU or micro-processor. The operations of the CPU and other electronic components are generally controlled at least in part by software programs running on the CPU (or other component). Such software programs may be stored in non-volatile memory, such as ROM, which can be integrated into the microcontroller itself, or provided as a separate component. The CPU may access the ROM to load and execute individual software programs as and when required. The microcontroller also contains appropriate communications interfaces (and control software) for communicating as appropriate with other devices in the body 10.

The body 20 further includes a cap 225 to seal and protect the far (distal) end of the e-cigarette 10. Typically, there is an air inlet hole provided in or adjacent to the cap 225 to allow air to enter the body 20 when a user inhales on the mouthpiece 35. The control unit or ASIC may be positioned alongside or at one end of the battery 210. In some embodiments, the ASIC is attached to a sensor unit 215 to detect an inhalation on mouthpiece 35 (or alternatively the sensor unit 215 may be provided on the ASIC itself). An air path is provided from the air inlet through the e-cigarette, past the airflow sensor 215 and the heater (in the vaporizer or cartomizer 30), to the mouthpiece 35. Thus, when a user inhales on the mouthpiece of the e-cigarette, the CPU detects such inhalation based on information from the airflow sensor 215.

At the opposite end of the body 20 from the cap 225 is the connector 25B for joining the body 20 to the cartomizer 30. The connector 25B provides mechanical and electrical connectivity between the body 20 and the cartomizer 30. The connector 25B includes a body connector 240, which is metallic (silver-plated in some embodiments) to serve as one terminal for electrical connection (positive or negative) to the cartomizer 30. The connector 25B further includes an electrical contact 250 to provide a second terminal for electrical connection to the cartomizer 30 of opposite polarity to the first terminal, namely body connector 240. The electrical contact 250 is mounted on a coil spring 255. When the body 20 is attached to the cartomizer 30, the connector 25A on the cartomizer 30 pushes against the electrical contact 250 in such a manner as to compress the coil spring in an axial direction, i.e. in a direction parallel to (co-aligned with) the longitudinal axis LA. In view of the resilient nature of the spring 255, this compression biases the spring 255 to expand, which has the effect of pushing the electrical contact 250 firmly against connector 25A of the cartomizer 30, thereby helping to ensure good electrical connectivity between the body 20 and the cartomizer 30. The body connector 240 and the electrical contact 250 are separated by a trestle 260, which is made of a non-conductor (such as plastic) to provide good insulation between the two electrical terminals. The trestle 260 is shaped to assist with the mutual mechanical engagement of connectors 25A and 25B.

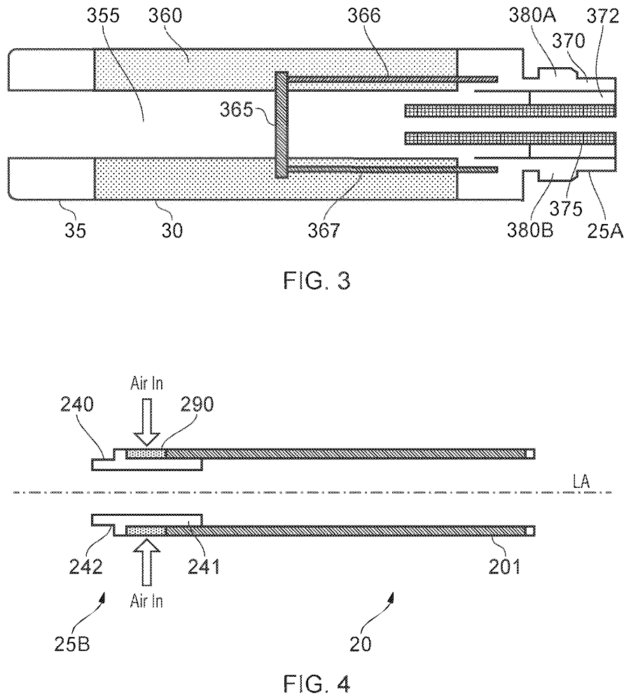

As mentioned above, a button 265, which represents a form of manual activation device 265, may be located on the outer housing of the body 20. The button 265 may be implemented using any appropriate mechanism, which is operable to be manually activated by the user—for example, as a mechanical button or switch, a capacitive or resistive touch sensor, and so on. It will also be appreciated that the manual activation device 265 may be located on the outer housing of the cartomizer 30, rather than the outer housing of the body 20, in which case, the manual activation device 265 may be attached to the ASIC via the connections 25A, 25B. The button 265 might also be located at the end of the body 20, in place of (or in addition to) cap 225. FIG. 3 is a schematic diagram of the cartomizer 30 of the e-cigarette 10 of FIG. 1 in accordance with some embodiments of the disclosure. FIG. 3 can generally be regarded as a cross-section in a plane through the longitudinal axis LA of the e-cigarette 10. Note that various components and details of the cartomizer 30, such as wiring and more complex shaping, have been omitted from FIG. 3 for reasons of clarity.

The cartomizer 30 includes an air passage 355 extending along the central (longitudinal) axis of the cartomizer 30 from the mouthpiece 35 to the connector 25A for joining the cartomizer to the body 20. A reservoir of liquid 360 is provided around the air passage 335. This reservoir 360 may be implemented, for example, by providing cotton or foam soaked in liquid. The cartomizer 30 also includes a heater 365 for heating liquid from reservoir 360 to generate vapor to flow through air passage 355 and out through mouthpiece 35 in response to a user inhaling on the e-cigarette 10. The heater 365 is powered through lines 366 and 367, which are in turn connected to opposing polarities (positive and negative, or vice versa) of the battery 210 of the main body 20 via connector 25A (the details of the wiring between the power lines 366 and 367 and connector 25A are omitted from FIG. 3).

The connector 25A includes an inner electrode 375, which may be silver-plated or made of some other suitable metal or conducting material. When the cartomizer 30 is connected to the body 20, the inner electrode 375 contacts the electrical contact 250 of the body 20 to provide a first electrical path between the cartomizer 30 and the body 20. In particular, as the connectors 25A and 25B are engaged, the inner electrode 375 pushes against the electrical contact 250 so as to compress the coil spring 255, thereby helping to ensure good electrical contact between the inner electrode 375 and the electrical contact 250.

The inner electrode 375 is surrounded by an insulating ring 372, which may be made of plastic, rubber, silicone, or any other suitable material. The insulating ring is surrounded by the cartomizer connector 370, which may be silver-plated or made of some other suitable metal or conducting material. When the cartomizer 30 is connected to the body 20, the cartomizer connector 370 contacts the body connector 240 of the body 20 to provide a second electrical path between the cartomizer 30 and the body 20. In other words, the inner electrode 375 and the cartomizer connector 370 serve as positive and negative terminals (or vice versa) for supplying power from the battery 210 in the body 20 to the heater 365 in the cartomizer 30 via supply lines 366 and 367 as appropriate.

The cartomizer connector 370 is provided with two lugs or tabs 380A, 380B, which extend in opposite directions away from the longitudinal axis of the e-cigarette 10. These tabs are used to provide a bayonet fitting in conjunction with the body connector 240 for connecting the cartomizer 30 to the body 20. This bayonet fitting provides a secure and robust connection between the cartomizer 30 and the body 20, so that the cartomizer and body are held in a fixed position relative to one another, with minimal wobble or flexing, and the likelihood of any accidental disconnection is very small. At the same time, the bayonet fitting provides simple and rapid connection and disconnection by an insertion followed by a rotation for connection, and a rotation (in the reverse direction) followed by withdrawal for disconnection. It will be appreciated that other embodiments may use a different form of connection between the body 20 and the cartomizer 30, such as a snap fit or a screw connection.

FIG. 4 is a schematic diagram of certain details of the connector 25B at the end of the body 20 in accordance with some embodiments of the disclosure (but omitting for clarity most of the internal structure of the connector as shown in FIG. 2, such as trestle 260). In particular, FIG. 4 shows the external housing 201 of the body 20, which generally has the form of a cylindrical tube. This external housing 201 may comprise, for example, an inner tube of metal with an outer covering of paper or similar. The external housing 201 may also comprise the manual activation device 265 (not shown in FIG. 4) so that the manual activation device 265 is easily accessible to the user.

The body connector 240 extends from this external housing 201 of the body 20. The body connector 240 as shown in FIG. 4 comprises two main portions, a shaft portion 241 in the shape of a hollow cylindrical tube, which is sized to fit just inside the external housing 201 of the body 20, and a lip portion 242 which is directed in a radially outward direction, away from the main longitudinal axis (LA) of the e-cigarette. Surrounding the shaft portion 241 of the body connector 240, where the shaft portion does not overlap with the external housing 201, is a collar or sleeve 290, which is again in a shape of a cylindrical tube. The collar 290 is retained between the lip portion 242 of the body connector 240 and the external housing 201 of the body, which together prevent movement of the collar 290 in an axial direction (i.e. parallel to axis LA). However, collar 290 is free to rotate around the shaft portion 241 (and hence also axis LA).

As mentioned above, the cap 225 is provided with an air inlet hole to allow air to flow when a user inhales on the mouthpiece 35. However, in some embodiments the majority of air that enters the device when a user inhales flows through collar 290 and body connector 240 as indicated by the two arrows in FIG. 4.

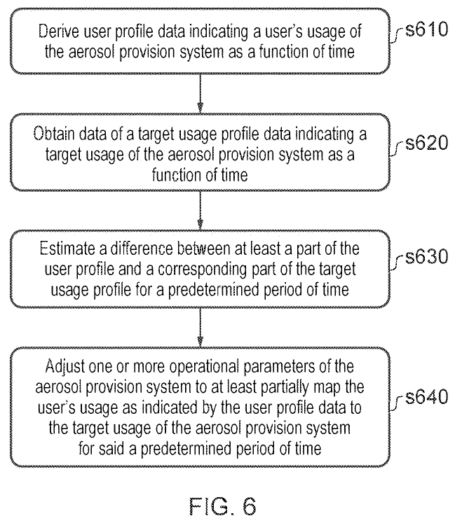

Referring now to FIG. 6, in an embodiment of the present disclosure it has been recognized that it may be desirable to modify a user's behavior, and specifically their usage of an aerosol provision system (electronic vapor provision system), by adjusting their behavior toward a target usage behavior. Furthermore, it is appreciated that whilst it may be possible to do this through reminders and notifications prompting the user to consciously change their behavior, this may be intrusive and inefficient. Accordingly, embodiments of the present disclosure seek to modify the effectiveness of the user's behavior by changing the response of the aerosol provision system in a manner that encourages user behavior closer to the target usage.

Accordingly, in an embodiment of the present disclosure a method of aerosol-generation comprises:

at s610, deriving user profile data indicating a user's usage of the aerosol provision system as a function of time;

at s620, obtaining data of a target usage profile data indicating a target usage of the aerosol provision system as a function of time;

at s630, estimating a difference between at least a part of the user profile and a corresponding part of the target usage profile for a predetermined period of time; and

at s640, adjusting one or more operational parameters of the aerosol provision system to at least partially map the user's usage as indicated by the user profile data to the target usage of the aerosol provision system for said a predetermined period of time.

At s610, the user profile data is generated/derived from usage data over repeated sampling periods of time. For example, a day may be broken up into 24 one-hour sampling periods, and a model of behavior in each period may be built up over successive days. Alternatively named days of the week may be broken up into 24 one-hour sampling periods, and a model of behavior for each period of each named day may be built up over successive weeks to build a model of behavior for each individual day. In this latter case, a basic model for a generic day using the first approach may be used to boot strap a refined model for each named day using the second approach in order to speed up derivation of the user profile data. Other approaches can also be considered, such as building a model for weekdays and weekends.

It will be appreciated that discrete one-hour periods are a non-limiting example. Other examples include overlapping sampling periods (for example one-hour periods provided every 30 minutes and overlapping neighboring periods by 15 minutes), and sampling periods of different length (for example discrete 20 minute periods, or half hour periods provided every 20 minutes and overlapping neighboring periods by five minutes). Optionally, sampling periods may be non-uniform; for example, periods may be determined by having on average an equal number of inhalation events in each period, as determined over time. In this case for example a weekday mid-afternoon period may be three hours long, as it typically only comprises a randomly timed e-cigarette break from work, whereas an early morning or early evening period may be 20 minutes long as the user takes the opportunity to use their aerosol provision device more frequently. Similarly, sampling periods may be driven by user behavior; for example, a period may correspond to a detected time dependent pattern or ‘session’, for example corresponding to a period of use above a first use-threshold, between periods of non-use or use below a second use-threshold. Clearly, also a mixture of such periods could be used, for example with overlapping or non-overlapping 1 hour, ½ hour or 20 minute periods by default, but one or more of these being replaced with frequency-driver periods (e.g. if the average number of inhalations within a default period is below a first threshold, then merge with the neighboring period, or if the average number of inhalations within a default period is above a second threshold, split the period), or with periods corresponding to a detected time dependent pattern that is apparent in the data.

In any event, user profile data for a given period may take the form of a rolling average of use based upon successive uses within that given period of time; this enables the usage to be characterized by persistent systematic or wholesale changes in behavior, whilst individual and isolated fluctuations in behavior do not contribute significantly to the overall profile.

The aspects of use that may contribute to the user profile data include but are not limited to one or more of the total active ingredient consumed within the period (for example nicotine), and the number, rate, duration and/or depth of inhalation actions within the period. It will be appreciated that the delivery of the total active ingredient within a given period is indicative of the user's demand within that period (which in turn may be driven by the structure of their day, both in terms of when they are able to use their aerosol provision system, e.g. out of core work hours, and in terms of when the wish to use their aerosol provision system, for example at periods of high stress, or in social situations).

Meanwhile parameters descriptive of how the user inhales to obtain that total active ingredient are indicative for example of the user's effectiveness in obtaining that total active ingredient; hence a large number of high frequency of shallow inhalations may be seen as less efficient than a lower number or lower frequency of deeper inhalations.

Optionally, the depth and/or duration of inhalation (where depth may be considered a function of airflow rate and duration or effective volume of air inhaled) may also be used to estimate the amount of total active ingredient inhaled by the user (as opposed to delivered by the aerosol provision system)—shallow inhalations are less likely to draw vapor/aerosol deep into the lungs, and so for an equivalent total amount of aerosol delivered over multiple shallow inhalations, less active ingredient may be delivered to the user then an equivalent volume of aerosol delivered over fewer deeper inhalations.

Hence, to a first approximation merely a number of inhalations may be used to estimate the amount of active ingredient delivered to the user, whilst to a second approximation a number and a depth/duration of inhalation may be used to refine the estimated amount of active ingredient delivered to the user.

In either case, user profile data for a given period of time may thus optionally comprise an indication of the amount of active ingredient notionally delivered to the user, and/or optionally data characterizing the type of inhalation behavior (number/rate/duration/depth) as discussed above.

Hence overall, the user profile data thus characterizes the likely/typical (average) usage behavior of the user for given periods of time.

Embodiments of the present disclosure then seek to modify this usage behavior, to steer the user towards a target behavior. Typically, the target behavior is to achieve a more uniform distribution of inhalation activity and delivery of active ingredient. There is a tendency for this to improve the users mood by reducing variability of the active ingredient in the users body and hence its pharmacological effects, and to assist with successful systematic reduction of levels of active ingredient, if this is desired by the user.

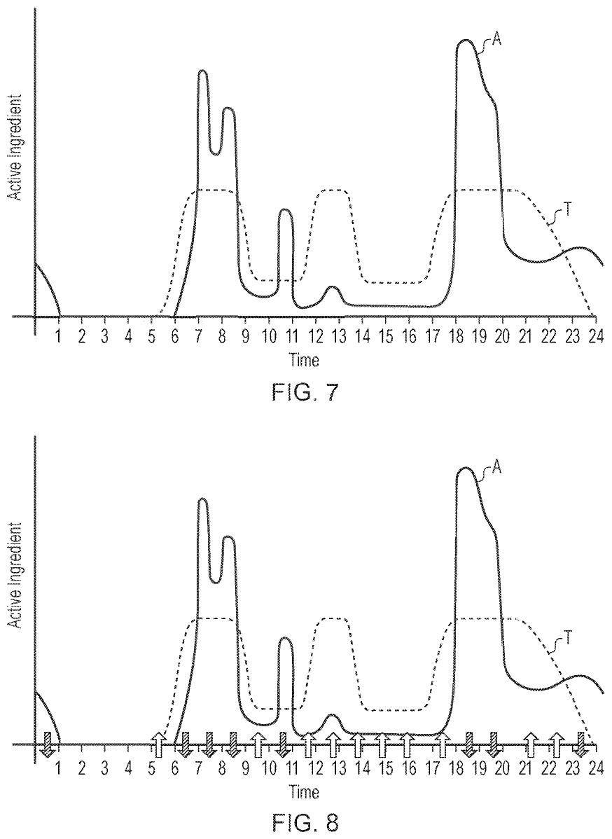

Referring now also to FIG. 7, this shows a graph with hours of the day on the x-axis and the amount of active ingredient consumed at a given time as an arbitrary scale on the y-axis. The graph shows an example usage behavior for a user as a solid line A, and a notional target usage as a dotted line T. In this case, the target usage may be one of a number of preset usage profiles, in this case corresponding to a working day profile.

Optionally, and as shown in this figure, the integral of the user's inhalation active ingredient and the target inhalation active ingredient are the same—in other words, this target represents a redistribution of the same total level of consumption in the day.

Hence, at s620, data from a target usage profile is obtained indicating a target usage of the aerosol provision system as a function of time.

It will be appreciated that any target profile may be created/selected and used. For example a user may create their own profile using a suitable graphical user interface on a mobile phone (for example, by drawing it with a finger), or may select from one of a plurality of profiles created by the developer of the app, or submitted to a pool of profiles by other users.

The app may provide different profiles according to different modes or intended uses; for example, the app may provide a daily, weekday/weekend or per named day profile make usage of the aerosol provision system more even throughout the day, or may provide a profile that reduces active ingredient intake towards the latter part of the day if the ingredient is a stimulant. Similarly the app may provide a succession of profiles as part of a behavior modification program, such as a program to reduce overall consumption of active ingredient over a period of time, for example over a series of weeks or months.

Alternatively or in addition, profiles may be selected based on a user's activities and their relative times for example, core working hours, ability to take breaks during those working hours, duration of lunch period, and the like. A profile may also be generated based on a questionnaire in the app or on a webpage logged into by the user asking questions such as those above. Alternatively or in addition, one or bespoke target profiles may be generated by applying a low-pass filter to the user's actual user profile data, whether for daily, weekday/weekend or per named day user profile data as described above. Again, such a profile may be the start point for a series of target profiles tending towards a preferred behavior, as described later herein. As noted above, s630 comprises estimating a difference between at least a part of the user profile and a corresponding part of the target usage profile for a predetermined period of time. It will be appreciated that for the purposes of explanation, FIG. 7 relates to usage in terms of overall consumption of active ingredient rather than in terms of number or frequency of inhalations (which is dealt with later herein).

In FIG. 7, it is apparent that the user has several intense usage sessions early in the morning before work. They typically also have a break at around 10:30. Meanwhile they do not appear to often use their aerosol provision system at lunchtime, for example due to eating at the desk. Possibly, as a result of this, they again have a prolonged intense session shortly after work. Subsequently after a lull they have a final session late in the evening/night.

The target profile aims to limit the intensity of each session, typically by broadening the sessions and making them more even. In particular, the target suggests having a varied break in the morning and afternoon (hence a uniform distribution of active ingredient consumption during these periods), and also offsetting some of the intense usage before and after work with more moderate usage during a lunch break. The target also encourages the user to graze on their aerosol provision system throughout the evening, tapering off towards bedtime.

In principle, this target profile gives the user a greater period of time in the day where the positive benefits of the active ingredient was avoiding intense usage sessions and significant late-night usage.

The estimated difference may therefore simply be a matter of subtracting the user's usage profile from the target usage profile (or vice versa). Optionally this may be done after scaling the target usage profile so that the integral of the two profiles is identical, or in the case of an active ingredient reduction or cessation program, the integral of the target usage profile may be a predetermined proportion of the user's actual usage profile.

In the case of FIG. 7, put crudely, there is excessive consumption between 11 PM and 1 AM, but consumption could be increased between 5 and 6 AM. Again, there is excessive consumption between 7 and 9 AM, whilst the consumption between 9 and 12 could be made more uniform. Consumption could be increased between 12 and 2 PM, and slightly increase on average between 2 and 5 PM. The consumption peak between 6 and 8 PM could then be distributed over a wider period between 5 and 10 PM by variously increasing and decreasing consumption during this period as required.

Consequently, s640 comprises adjusting one or more operational parameters of the aerosol provision system to at least partially map the user's usage as indicated by the user profile data to the target usage of the aerosol provision system for said a predetermined period of time.

Again, as noted above FIG. 7 relates to overall consumption of the active ingredient. Consequently, for example, provision system can be adjusted to modify the amount of active ingredient delivered for individual puffs during the course of the day so that the user's overall consumption is closer to the target than their original user profile.

Referring now also to FIG. 8, this shows the same original user profile data and targeted, together with an arrows indicating of whether the amount of active ingredient delivered for individual puffs is increased or decreased during a given period of time. It will be appreciated that the arrows are a crude indication, and that the increase or decrease may for example be proportional to the actual difference between the user and target profiles at any given time, optionally subject to a capped maximum.

This increase or decrease for example may be achieved by adjusting operational parameters change the amount of active ingredient inhaled, for example either by changing the amount of air inhaled by altering airflow within the aerosol provision system, or more typically by altering the amount of active ingredient delivered per unit volume of air inhaled.

Hence, for example the amount of active ingredient may be increased by increasing the temperature of the heater of the aerosol provision system to increase the amount of vapor generated per unit volume of air. Meanwhile, the amount of active ingredient may be decreased by unit volume of air for example by either reducing the temperature of the heater, or introducing a duty cycle so that the heater operates intermittently (as a non-limiting example, the heater may be on for between one 100^{th} and one 10^{th} of a second every 10^{th} of a second, thereby generating between 10% and 100% of normal output depending on the timings).

Hence, for example in this way the user will receive a larger than average amount of active ingredient during periods where actual usage is below the target, and will receive a less than average amount of active ingredient during periods where the actual usage is above the target, all else being equal.

As noted previously, the increase or decrease may relate to only a percentage of the difference between the actual and target profiles (for example 25%, 50%, 75%, 100%). This modification may slowly increase over time so that the user is gently led towards consumption of active ingredient at the target profile's distribution.

Notably, providing more active ingredient where the target is above actual usage can be considered to reward the user for changing behavior toward the target, whilst providing less active ingredient where actual usage is above the target can be considered in part to discourage the user from continuing this behavior, and also in part mitigating the effects of this behavior.

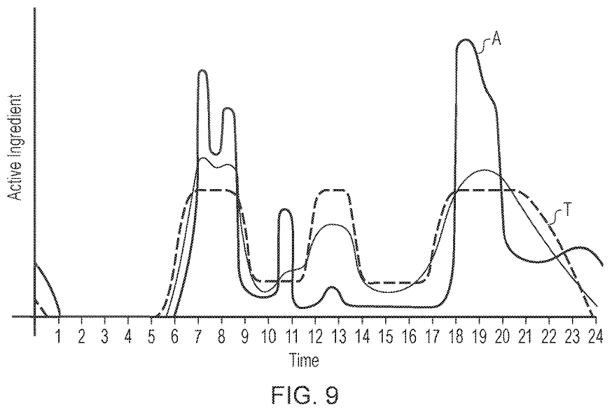

Subsequently, referring now to FIG. 9, this depicts a modified usage by the user as a long-dashed (red) line. Following exposure to the modified delivery of active ingredient by their aerosol provision system, the user has been encouraged to use their aerosol provision system slightly earlier in the morning until slightly later, but less intensively overall. Meanwhile the user has started to vary their usage of the system mid-morning, and increase usage around lunchtime. They have also been encouraged to start use of the device earlier after work, and as a result are using it much less intensely in the early evening and their usage into the late evening has been significantly curtailed. Overall, their total usage is roughly the same as before, but more evenly distributed. In this way, the user has been nudged towards a more uniform distribution of use and absorption of active ingredient by their body, by the combination of rewarding use at times which are underutilized according to the target profile, and reducing the effectiveness of use at times where usage is higher than the target profile.

Subsequently, the user may continue to converge on the target profile in response to the variation in effectiveness of inhalation caused by the amount of active ingredient delivered responsive to the difference between usage profile data and target data. However optionally the target profile itself may be refined over time (for example by reducing the integral of the target profile as part of a reduction or cessation program), for example once the overall difference between the user profile data and target data is less than a threshold amount.

Optionally, when a target usage profile is used in the above-described manner to encourage a change of behavior, the user's profile data is updated more quickly, for example by using a shorter rolling average, to reflect relatively quick changes behavior may be caused by the modifications to inhalation effectiveness described above.

In the examples of FIGS. 7, 8, and 9, the differences between the target and use were evaluated fairly continuously over the course of a day. However more generally in addition to estimating and adjusting over the course of a day, the period may be based on when the aerosol provision system is disconnected from a charger (for example to indicate the start of use in a day, or to relate an aspect of inhalation behavior or delivery modification to battery capacity). Similarly, the start of use in a day can be signified by the first inhalation of the day, or from the current time (for example when a user initiates a control program; for example, they may only wish to modify their inhalation behavior during evenings or weekends).

Again whilst examples of FIGS. 7, 8, and 9 show evaluation continuously over the day, as noted previously the predetermined period of time for estimation and adjustment may correspond in length to a detected time-dependent pattern in the user profile data, or indeed to a detected time-dependent pattern in the target usage profile. Period may relate to the whole day itself where only the overall consumption level is being controlled. However, typically the predetermined period will correspond to the sampling periods of the user profile data as described previously.

The above discussion of modifying the effectiveness of inhalation to encourage increase or decrease of consumption of active ingredient toward the target effectively assumes the same distribution of individual inhalation actions during the day, whilst modifying the delivery of active ingredient during these individual inhalation actions.

However it will be appreciated that the user's pattern of inhalation may also change, for example with more inhalations occurring during periods previously underutilized with respect to the target profile, and potentially fewer inhalations during periods that previously saw more intense use than in the target profile.

Accordingly, adjustment may comprise at least partially mapping the user's usage as indicated by the user profile data to the target usage of the aerosol provision system, where the mapping distributes the total delivered active ingredient indicated by the target usage profile across the user's usage for the predetermined period as indicated by the user profile data. In this case, it is assumed that the user profile data also comprises numbers and frequency of inhalations per sample period, and will try to divide the total modified amount of active ingredient to be delivered within the period between the expected number of inhalations for that period.

Optionally, the technique can respond to the actual inhalation behavior during the time corresponding to the current sample period, so that for example if the user inhales more frequently, the amount of active ingredient delivered can either be reduced per inhalation to maintain the overall target for that period, and/or can be stopped if the target for that period is reached (or a predetermined proportion over the target, to allow for user variability). Alternatively, or in addition the system can selectively or preferentially deliver active ingredient during inhalations corresponding to those in the target schedule, to encourage a target usage pattern, as described later herein.

With respect to the number or frequency of inhalations, optionally a target user profile may also comprise a target number or frequency of inhalations within a predetermined period of time, which may be thought of as the cadence of inhalation. A crude distinction for example may be between a ‘bursting’ behavior where the user inhales frequently within a 10 or 15 minute period and then substantially ceases inhalation for 30 minutes, and a grazing behavior where the user inhales less frequently but substantially uniformly for an hour.

Accordingly, adjustment may comprise at least partially mapping the user's usage as indicated by the user profile data to the target usage of the aerosol provision system, where the mapping distributes the delivery of active ingredient within user inhalations responsive to a schedule of inhalations within the target usage of the aerosol provision system. Hence, for example inhalations by the user closer to the target timing may deliver more active ingredient than inhalations between target timings. Hence for example if the user has a period where the intensively use the aerosol provision system and hence perform inhalation actions twice as frequently as the target usage, then the aerosol provision system may only deliver active ingredient on alternate inhalations.

In practice, rather than relying upon specific timing events in the target profile, it is more likely that a preferred frequency is stipulated, and user inhalation events close to the frequency will receive an amount of active ingredient similar to that described previously herein depending upon the difference between the user profile and the target profile, whilst user inhalation events above the frequency may receive a proportionally reduced amount of active ingredient, or may receive an amount of active ingredient dependent upon where in the target frequency cycle inhalation takes place.

It will be appreciated that the case of inhalation timings, the user may be provided with additional feedback to assist them, for example in terms of a traffic light system for a light on the aerosol provision system indicating being at (green) close to (amber) or in between (red) preferred inhalation timings. Similarly, a graphical feedback could be provided by an accompanying mobile phone or similar remote device, or indeed a display of the aerosol provision system itself.

In addition to nudging the user toward the target amount of active ingredient during a predetermined period, and alternatively or in addition nudging the user toward a target cadence of inhalations during a predetermined period, embodiments of the techniques described herein can nudge user toward a target depth or duration of inhalation, for example to reduce stress-related inhalation behavior at certain times of day. For example, an inhalation characterized by a sharp deep initial inhalation may be characteristic of stress, and consequently a deep (high-volume) but short duration inhalation may be indicative of undesirable stress or an undesirable use of the aerosol provision system in response to that stress.

Accordingly, adjustment may comprise at least partially mapping the user's usage as indicated by the user profile data to the target usage of the aerosol provision system, where the mapping distributes the delivery of active ingredient during a respective user inhalation responsive to the difference between an expected inhalation duration indicated by the user profile data and a corresponding target inhalation duration.

An inhalation delivery profile can be modified for example to start delivering aerosol after a short delay following the initial triggering intake of breath at the start of an inhalation action, so that the user maximizes their intake of active ingredient by inhaling more slowly. Alternatively or in addition, the activation threshold for the heater may be increased so that aerosol is only delivered once a sufficiently deep intake of breath is detected; this may be used to help a user train themselves by deliberately using an artificially strong inhalation to signify a conscious choice of following a target timing and/or usage regime.

It will be appreciated that any of the above techniques may be used in any suitable combination. Hence as non-limiting examples, a target profile may specify an amount of active ingredient per predetermined period, and the predetermined periods within the original target profile, together with the target amounts, may optionally be converted to overlapping periods, non-uniform periods and/or the like if these have been used to characterize the user's profile. In either case, the target profile may optionally also specify a target frequency of use per predetermined period, and/or optionally a predetermined inhalation profile (for example designed to discourage excessively deep inhalations at certain points of the day). The aerosol provision system or a companion app on a mobile phone providing controls to the aerosol provision system can then calculate a combination of expected rates of delivery of active ingredient within given predetermined periods, optionally additionally based on relative frequency of use of the aerosol provision system, relative patterns of use of the aerosol provision system, and relative depth/volume of inhalation of the aerosol provision system within these periods. It will be appreciated that the above techniques potentially modify the behavior of the aerosol provision system to a significant extent (with the intention of changing the behavior of the user in turn), and this could cause frustration, annoyance, or lack of trust in the user if the user is not given knowledge of this process and does not give active consent to its use.

Accordingly, in embodiments of the present disclosure the method comprises receiving from a user interface an indication from the user to commence mapping (i.e. actively adjusting the behavior of the aerosol provision system). The aerosol provision system or the user interface of a companion app may also provide the option of suspending/overriding the mapping scheme, for example so that the user can more temporarily use the aerosol provision system or intensively for any particular reason. Optionally during such a period the user profile is not updated so that this isolated change of behavior does not affect the user profile and hence the subsequent mapping process.

Similarly, in principle the user can also turn the mapping process off. Meanwhile if the user has no intention of taking advantage of the mapping/adjustment feature at any point in the near future, then in principle the user could also specify that a user profile is not generated for them.

The above embodiments are intended to nudge a user towards a more beneficial pattern of use of their aerosol provision system, by modifying the effectiveness of its use as a function of the difference between the user's actual pattern of use any desired target use.

As noted previously, this pattern may be similar every day, or may change between weekdays and weekends, or may differ each day of the week, and separate user profiles can be created and used to accommodate these differences.

However it will also be appreciated that behavior and usage can change based on where the user is; for frequent changes of location, such as work and home, these can in principle be accommodated by the timings of behavior within user profiles for weekday and weekend profiles.

However for people who work irregular shifts such timings may not be possible, and so a location dependent profile may be more appropriate, with the relevant location sensitive user profile relating for example to an eight-hour shift rather than a 24-hour day. Corresponding target profiles may be provided for such shiftwork, or alternatively that part of a conventional target profile corresponding to normal office hours may be used as a target shift profile. A work location may be specified by the user via a user interface of a companion app on a mobile phone for example, using their current GPS location at work. Meanwhile a home location may be specified in a similar manner.

Similarly, users tend to change their behavior when on holiday. Accordingly, mapping/alteration of behavior of the aerosol provision system may be suspended when the user travels more than a predetermined distance from home, or has not returned home within a 24-hour period, or is detected to be in a different country to their in country, etc.; alternatively or in addition, a holiday user profile or profiles may be generated and used in a similar manner to those described previously herein, for example using a weekend profile to boot strap a holiday profile with data, and at least initially using a comparatively short rolling average to enable refinement of the model.

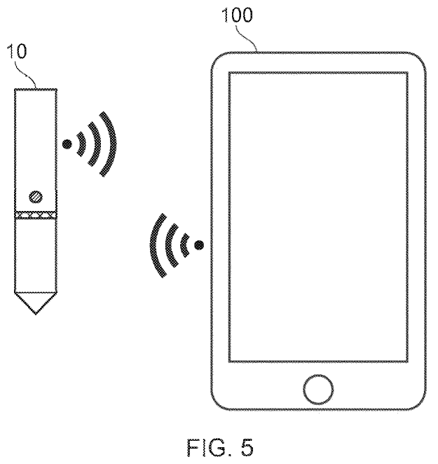

It will be appreciated that the above methods and techniques may be carried out on conventional hardware suitably adapted as applicable by software instruction or by the inclusion or substitution of dedicated hardware. Typical hardware is illustrated in FIG. 5, with an aerosol provision system/electronic vapor provision system 10 in communication with a remote device such as a mobile phone 100 operating under software instruction (for example a companion app) operable to implement the techniques described herein.

The extent to which each of the aerosol provision system and the companion app implement operations described above can vary from the aerosol provision system being limited to transmitting raw usage data and receiving control data from the mobile phone and the mobile phone performing the remaining operations, to the aerosol provision system performing most or all of the operations, and the mobile phone being limited to a user interface. Optionally where the aerosol provision system comprises a suitable user interface of its own, then associated device may not be necessary.

It will be appreciated that instead of a local communication link to a mobile phone, the aerosol provision system may similarly operate with a remote server via a Wi-Fi or mobile data connection. In this case, if user interactions beyond the capabilities of the aerosol provision system itself are required, then these may be accessed via a web page supplied by the server.

In any case, the required adaptation to existing parts of a conventional equivalent device may thus be implemented in the form of a computer program product comprising processor implementable instructions stored on a non-transitory machine-readable medium such as a floppy disk, optical disk, hard disk, solid state disk, PROM, RAM, flash memory or any combination of these or other storage media, or realized in hardware as an ASIC (application specific integrated circuit) or an FPGA (field programmable gate array) or other configurable circuit suitable to use in adapting the conventional equivalent device. Separately, such a computer program may be transmitted via data signals on a network such as an Ethernet, a wireless network, the Internet, or any combination of these or other networks.

Accordingly, an aerosol provision system may be provided at is configured to generate aerosol from an aerosol-generating material for user inhalation, which comprises a computer configured to implement the operations of the method described previously herein, namely to derive user profile data indicating a user's usage of the aerosol provision system as a function of time, obtain data of a target usage profile indicating a target usage of the aerosol provision system as a function of time, estimate a difference between at least a part of the user profile and a corresponding part of the target usage profile for a predetermined period of time, and adjust one or more operational parameters of the aerosol provision system to at least partially map the user's usage as indicated by the user profile data to the target usage of the aerosol provision system for said a predetermined period of time.

Meanwhile it will be apparent to a person skilled in the art that such apparatus may implement variations in the above method corresponding to operation of the various embodiments of the method and/or apparatus as described and claimed herein are considered within the scope of the present disclosure, including but not limited to where:
the predetermined period of time starts at one selected from the list consisting of a calendar day, a disconnection of the aerosol provision system from a charger, a first inhalation in a day and the current time;
the predetermined period of time is one selected from the list consisting of a period corresponding in length to a detected time dependent pattern in the user profile data, a period corresponding in length to a detected time dependent pattern in the target usage profile, a sampling period of the user profile data, an hour, and a day;
the operational parameters are adjusted to change the amount of an active ingredient delivered per unit volume of air inhaled;
the computer is configured to at least partially map the user's usage as indicated by the user profile data to the target usage of the aerosol provision system, where the mapping distributes the total delivered active ingredient indicated by the target usage profile across the user's usage for the predetermined period as indicated by the user profile data;
the computer is configured to at least partially map the user's usage as indicated by the user profile data to the target usage of the aerosol provision system, where the mapping distributes the delivery of active ingredient within user inhalations responsive to a schedule of inhalations within the target usage of the aerosol provision system;
the computer is configured to at least partially map the user's usage as indicated by the user profile data to the target usage of the aerosol provision system, where the mapping distributes the delivery of active ingredient during a respective user inhalation responsive to the difference between an expected inhalation duration indicated by the user profile data and a corresponding target inhalation duration;
the computer is configured to receive from a user interface an indication from the user to commence mapping; and
the operations of the computer are located within one or more selected from the list consisting of the aerosol provision system, a remote server operable to communicate with the aerosol provision system, a mobile computing device operable to communicate with the aerosol provision system, and a remote server operable to communicate with a mobile computing device operable to communicate with the aerosol provision system.

Finally, referring again to FIG. 1, it will be appreciated from the discussion previously herein that the EVPS may be a self-contained unit (commonly referred to as an e-cigarette, even if the device itself does not necessarily conform to the shape or dimensions of a conventional cigarette). Such an e-cigarette may comprise an airflow measuring means, a processing means and optionally one or more feedback means such as haptic, audio and/or light/display means.

Alternatively, referring to FIG. 5, an EVPS system may comprise two components, such as an e-cigarette 10 and a mobile phone or similar device (such as a tablet) 100 operable to communicate with the e-cigarette (for example to at least receive data from the e-cigarette), for example via Bluetooth.

The mobile phone may then comprise the processing means and one or more feedback means such as haptic, audio and/or light/display means, alternatively o in addition to those of the e-cigarette.

Optionally an EVPS system may comprise an e-cigarette 10 operable to communicate with a mobile phone 100, in which the mobile phone stores one or more parameters or other data (such as data characteristic of one or more aspects of usage by the user) for the EVPS, and receives such parameters/data from the e-cigarette. The phone may then optionally perform processing on such parameters/data and either return processed data and/or instructions to the EVPS, display a result to the user (or perform another action) or forward processed and/or unprocessed parameters/data on to a remote server.

Optionally the mobile phone or the EVPS itself may be operable to wirelessly access data associated with an account of the user at such a remote server. In a variant embodiment of the disclosure, a first EVPS of a user may communicate some or all of its user settings to another EVPS. The user settings may comprise settings related to an implementation of the above disclosed methods, such as data characteristic of user behavior, and/or data relating to modification of the EVPS operation.

Such data may be relayed between devices either directly (e.g. via a Bluetooth or near-field communication) or via one or more intermediary devices, such as a mobile phone owned by the user of the two devices or a server on which the user has an account. In this way, a user may easily share the data from one device to another, for example if the user has two EVPS devices, or if the user wishes to replace one EVPS device with another without losing accumulated personalization data.

Optionally in this embodiment, where the second EVPS differs in type from the first EVPS (for example by having a different default power level, or heating efficiency), then a conversion factor or look-up table for converting operational parameters from the first EVPS to the second EVPS may be employed. This may be provided in software or firmware of the second EVPS, and identify the first EVPS and hence the appropriate conversions when making direct communication (or where data is relayed without change via an intermediary such as a phone). Alternatively or in addition an app on the phone may provide the conversion, optionally downloading the relevant conversions in response to the identity of the first and second EVPS. Again, alternatively or in addition a remote server may provide the conversion, in response to the identity of the first and second EVPS as associated with a user's account.

The foregoing discussion discloses and describes merely exemplary embodiments of the present disclosure. As will be understood by those skilled in the art, the present disclosure may be embodied in other specific forms without departing from the essential characteristics thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. An aerosol provision system configured to generate aerosol from an aerosol generating material for user inhalation, the aerosol provision system comprising:
a computer configured to:
derive user profile data indicating a user's usage of the aerosol provision system as a function of time;
obtain data of a target usage profile indicating a target usage of the aerosol provision system as a function of time;
estimate a difference between at least a part of the user profile and a corresponding part of the target usage profile for a predetermined period of time; and
adjust one or more operational parameters of the aerosol provision system to at least partially map the user's usage as indicated by the user profile data to the target usage of the aerosol provision system for the predetermined period of time.

2. The aerosol provision system according to claim 1, in which the predetermined period of time starts at one of:
i. a calendar day;
ii. a disconnection of the aerosol provision system from a charger;
iii. a first inhalation in a day; or
iv. the current time.

3. The aerosol provision system according to claim 1, in which the predetermined period of time is one of:
i. a period corresponding in length to a detected time dependent pattern in the user profile data;
ii. a period corresponding in length to a detected time dependent pattern in the target usage profile;
iii. a sampling period of the user profile data;
iv. an hour; or
v. a day.

4. The aerosol provision system according to claim 1, in which the one or more operational parameters are adjusted to change the amount of an active ingredient delivered per unit volume of air inhaled.

5. The aerosol provision system according to claim 4, in which the computer is configured to:
at least partially map the user's usage as indicated by the user profile data to the target usage of the aerosol provision system, wherein the mapping distributes the total delivered active ingredient indicated by the target usage profile across the user's usage for the predetermined period of time as indicated by the user profile data.

6. The aerosol provision system according to claim 1, in which the computer is further configured to:
at least partially map the user's usage as indicated by the user profile data to the target usage of the aerosol provision system, wherein the mapping distributes delivery of an active ingredient within user inhalations responsive to a schedule of inhalations within the target usage of the aerosol provision system.

7. The aerosol provision system according to claim 1, in which the computer is further configured to:
at least partially map the user's usage as indicated by the user profile data to the target usage of the aerosol provision system, wherein the mapping distributes delivery of an active ingredient during a respective user inhalation responsive to the difference between an expected inhalation duration indicated by the user profile data and a corresponding target inhalation duration.

8. The aerosol provision system according to claim 1, in which the computer is further configured to:
receive from a user interface an indication from the user to commence mapping.

9. The aerosol provision system of claim 1, in which the operations of the computer are located within one or more of:
i. the aerosol provision system;
ii. a remote server operable to communicate with the aerosol provision system;
iii. a mobile computing device operable to communicate with the aerosol provision system; and
iv. a remote server operable to communicate with a mobile computing device operable to communicate with the aerosol provision system.

10. A method of aerosol generation, comprising:
deriving user profile data indicating a user's usage of an aerosol provision system as a function of time;
obtaining data of a target usage profile indicating a target usage of the aerosol provision system as a function of time;
estimating a difference between at least a part of the user profile data and a corresponding part of the target usage profile for a predetermined period of time; and
adjusting one or more operational parameters of the aerosol provision system to at least partially map the user's usage as indicated by the user profile data to the target usage of the aerosol provision system for the predetermined period of time.

11. The method of claim 10, in which the predetermined period of time starts at one of:
i. a calendar day;
ii. a disconnection of the aerosol provision system from a charger;
iii. a first inhalation in a day; or
iv. the current time.

12. The method of claim 10, in which the predetermined period of time is one of:
i. a period corresponding in length to a detected time dependent pattern in the user profile data;
ii. a period corresponding in length to a detected time dependent pattern in the target usage profile;
iii. a sampling period of the user profile data;
iv. an hour; or
v. a day.

13. The method of claim 10, in which the one or more operational parameters are adjusted to change the amount of an active ingredient delivered per unit volume of air inhaled.

14. The method of claim 13, in which adjusting one or more operational parameters of the aerosol provision system further comprises at least partially mapping the user's usage as indicated by the user profile data to the target usage of the aerosol provision system, wherein the mapping distributes the total delivered active ingredient indicated by the target usage profile across the user's usage for the predetermined period of time as indicated by the user profile data.

15. The method of claim 10, in which adjusting one or more operational parameters of the aerosol provision system further comprises at least partially mapping the user's usage as indicated by the user profile data to the target usage of the aerosol provision system, wherein the mapping distributes delivery of an active ingredient within user inhalations responsive to a schedule of inhalations within the target usage of the aerosol provision system.

16. The method of claim 10, in which the adjustment step comprises at least partially mapping the user's usage as indicated by the user profile data to the target usage of the aerosol provision system, where the mapping distributes delivery of an active ingredient during a respective user inhalation responsive to the difference between an expected inhalation duration indicated by the user profile data and a corresponding target inhalation duration.

17. The method of claim 10, further comprising the step of receiving from a user interface an indication from the user to commence mapping.

18. The method of claim 10, in which implementation of the method steps are carried out by one or more of:
i. an aerosol provision system;
ii. a remote server operable to communicate with the aerosol provision system;
iii. a mobile computing device operable to communicate with the aerosol provision system; and
iv. a remote server operable to communicate with a mobile computing device operable to communicate with the aerosol provision system.

19. A non-transitory computer-readable storage medium storing a computer program comprising computer executable instructions adapted to cause a computer system to perform the method of claim 1.
